(19) United States
(12) Patent Application Publication
(10) Pub. No.: US 2009/0326584 A1
(43) Pub. Date: Dec. 31, 2009

(54) Spinal Dynamic Stabilization Rods Having Interior Bumpers

(76) Inventors: Michael Andrew Slivka, Taunton, MA (US); Ian C. Burgess, Barrington, RI (US); Alexander Grinberg, Newton, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/163,704

(22) Filed: Jun. 27, 2008

(51) Int. Cl. A61B 17/70

(52) U.S. Cl. 606/261; 606/246; 606/278

(57) ABSTRACT

A dynamic stabilization device for use in the human spine, comprising i) a hollow, closed-end cylinder component that is configured for attachment to a first pedicle screw, ii) a solid rod component that is configured on one end to slide freely within the hollow cylinder component, and configured at the other end for attachment to a second pedicle screw and iii) an elastomer component contained within the hollow cylinder and located between the closed end of the hollow cylinder and the end of the solid rod component.

### BACKGROUND OF THE INVENTION

The vertebrae in a patient's spinal column are linked to one another by the disc and the facet joints, which control movement of the vertebrae relative to one another. Each vertebra has a pair of articulating surfaces located on the left side, and a pair of articulating surfaces located on the right side, and each pair includes a superior articular surface, which faces upward, and an inferior articular surface, which faces downward. Together the superior and inferior articular surfaces of adjacent vertebra form a facet joint. Facet joints are synovial joints, which means that each joint is surrounded by a capsule of connective tissue and produces a fluid to nourish and lubricate the joint. The joint surfaces are coated with cartilage allowing the joints to move or articulate relative to one another.

Diseased, degenerated, impaired, or otherwise painful facet joints and/or discs can require surgery to restore function to the three joint complex. Damaged, diseased levels in the spine were traditionally fused to one another. While such a technique may relieve pain, it effectively prevents motion between at least two vertebrae. As a result, additional stress may be applied to the adjoining levels, thereby potentially leading to further damage.

More recently, techniques have been developed to restore normal function to the facet joints. One such technique involves covering the facet joint with a cap to preserve the bony and articular structure. Capping techniques, however, are limited in use as they will not remove the source of the pain in osteoarthritic joints. Caps are also disadvantageous as they must be available in a variety of sizes and shapes to accommodate the wide variability in the anatomical morphology of the facets. Caps also have a tendency to loosen over time, potentially resulting in additional damage to the joint and/or the bone support structure containing the cap.

Other techniques for restoring the normal function to the posterior element involve arch replacement, in which superior and inferior prosthetic arches are implanted to extend across the vertebra typically between the spinous process. The arches can articulate relative to one another to replace the articulating function of the facet joints. One drawback of current articulating facet replacement devices, however, is that they require the facet joints to be resected. Moreover, alignment of the articulating surfaces with one another can be challenging.

Accordingly, there remains a need for improved systems and methods that are adapted to mimic the natural function of the facet joints.

Traditional spine fusion may result in iatrogenic instability at adjacent spine levels and subsequently require additional surgery to fuse more levels. Stabilization using more dynamic rods with traditional pedicle screw instrumentation may improve surgical outcomes and reduce additional surgeries for adjacent level degeneration.

U.S. Pat. No. 5,540,688 (Navas) discloses an intervertebral stabilization device, made in the form of a damper adapted to resist elastically, on the one hand, an elongation and, on the other hand, an axial compression without buckling, as well as of at least two implants anchored on two adjacent vertebrae. U.S. Pat. No. 5,672,175 (Martin) discloses. a dynamic implanted spinal orthosis which preserves at least in part the natural physiological mobility of the vertebrae while effecting and maintaining a correction of the relative positions of the vertebrae without osteosynthesis, graft or fusion, comprising anchoring components fixed to the vertebrae and holding means associated with the anchoring components for holding the vertebrae with respect to each other in the corrected position, the holding means comprise an elastic return device for exerting elastic return forces, the orientation and magnitude of which are determined for holding the vertebrae in the corrected position against natural deforming forces for reducing the forces exerted on the vertebrae while preserving their mobility; also a procedure for maintaining a correction of the positions of the vertebrae for treating a deformation of the spine.

U.S. Pat. No. 5,375,823 (Navas) discloses an improved damper, of the type comprising elements for progressively resisting, in exponential manner, the advance of a piston under the effect of a force of axial compression, which functions as a stop opposing any displacement of the piston beyond a predetermined value, in an intervertebral stabilization device.

U.S. Pat. No. 5,562,737 (Graf) discloses An extra-discal intervertebral prosthesis comprising at least a partially closed, elongated body including a compression chamber having an elastic block at one end. The block has a free face abutted by a ball joint associated with a first of two fixation means engagable in spaced vertebrae of a patient.

U.S. Pat. No. 6,241,730 (Alby) discloses an intervertebral link device including at least one damper element constituted by a cage and a pin designed to be connected to bone anchor elements. The pin being engaged in a housing of the cage and being fitted with two elastically deformable members operating in opposition to an applied traction force or compression force. The damper element includes a pin that is mounted inside the cage by a joint allowing multidirectional relative pivoting between the pin and the cage, at least about the axes contained in a plane perpendicular to the pin and angular abutment between the cage and the pin enabling the multidirectional relative pivoting to be limited in amplitude to a determined value of about 4°

US Patent Publication No. 2003/0220643 (Ferree) discloses an apparatus for inhibiting full extension between upper and lower vertebral bodies, thereby preventing pain and other complications associated with spinal movement. In the preferred embodiment, the invention provides a generally transverse member extending between the spinous processes and lamina of the upper and lower vertebral bodies, thereby inhibiting full extension. Various embodiments of the invention may limit spinal flexion, rotation and/or lateral bending while preventing spinal extension. In the preferred embodiment, the transverse member is fixed between two opposing points on the lower vertebral body using pedicle screws, and a cushioning sleeve is used as a protective cover. The transverse member may be a rod or cable, and the apparatus may be used with a partial or full artificial disc replacement. To control spinal flexion, rotation and/or lateral bending one or more links may be fastened to an adjacent vertebral body, also preferably using a pedicle screw. Preferably a pair of opposing links are used between the upper and lower vertebral bodies for such purposes. Alternative embodiments use stretchable elements with or without a transverse member.

US Patent Publication No. 2004/0049189 (Le Couedic) discloses a connecting member for maintaining the spacing between at least two anchor members screwed into vertebrae. It comprises two rigid rod-forming parts made of a first material and each having a fixing, first portion adapted to be fixed into an anchor member and a fastening, second portion, said rods being aligned with each other and said fastening portions facing each other, and a connecting body made of a second material which is more elastically deformable than said first material and which interconnects said rigid parts by means of the facing fastening portions so that said connecting body is able to deform elastically, whereby the vertebrae, which are held spaced from each other, are movable relative to each other.

US Patent Publication No. 2005/0203519 (Harms) discloses a rod-shaped element for use in spinal or trauma surgery, having a first section for connecting to a first bone anchoring element and a second section for connecting to a second bone anchoring element. The rod-shaped element also includes a first elastic flexible element that is capable of elastic deformation when a force acts on it transverse to the rod axis. The first section and the second section are capable of shifting relative to each other in the direction of the rod axis. In a stabilization device for use in spinal or trauma surgery, the rod-shaped element allows for a controlled motion of the parts to be stabilized relative to each other so flexural motion is adjusted separately from the adjustment of the mobility in axial direction

US Patent Publication No. 2005/0288670 (Panjabi) disclosesspine stabilization devices, systems and methods in which a single resilient member or spring is disposed on an elongate element that spans two attachment members attached to different spinal vertebrae. The elongate element passes through at least one of the two attachment members, permitting relative motion therebetween, and terminates in a stop or abutment. A second resilient member is disposed on the elongate element on an opposite side of the sliding attachment member, e.g., in an overhanging orientation. The two resilient members are capable of applying mutually opposing urging forces, and a compressive preload can be applied to one or both of the resilient members.

US Patent Publication No. 2006/0265074 (Krishna) discloses a lumbar disc prosthesis including a pair of disc members. The first member of the disc pair has a vertebral disc contact surface and a recessed portion on an opposing surface thereof. The second member of the disc pair has a vertebral disc contact surface and a protruding portion on an opposing surface thereof. The protruding portion of the second member engages with the recessed portion of the first member in use. Each of the first and second disc members are provided with at least three sections; a middle section and two end sections. The recessed and protruding portions are provided in the middle section of the respective disc members and each of the two end sections have a narrowing taper towards the ends of the disc members. The facet joint prosthesis includes a first member for attachment to a first posterior lumbar disc in use and a second member for attachment to a second posterior lumbar disc in use. At least a part of the first member is telescopically mounted in at least a part of the second member in use.

### SUMMARY OF THE INVENTION

The present invention relates to the use of internal elastomeric bumpers within a dynamic stabilization (PDS) system.

This invention comprises dynamic stabilizing rods that can be connected to pedicle screws and fixed to the spine. In a preferred embodiment, the rod comprises a hollow, closed-end cylinder component that is configured for attachment to a first pedicle screw, a solid rod component that is configured on one end to slide freely within the hollow cylinder component, and configured at the other end for attachment to a second pedicle screw and an elastomer component contained within the hollow cylinder and located between the closed end of the hollow cylinder and the end of the solid rod component.

Therefore, in accordance with the present invention, there is provided a dynamic stabilization device comprising:
a) a first hollow cylinder having an open end, an intermediate annular portion and a closed end, the closed end defining an inner surface, the intermediate annular portion defining an outer annular surface and a first inner annular surface,
b) a first rod having an outer diameter, a first end having a first end surface, and a second end, the first end of the first rod being slidably received within the inner annular surface of the first hollow cylinder, and
c) a first elastomeric bumper having a first end and a second end,
   wherein the first end of the first elastomeric bumper is attached to the inner surface of the closed surface of the first hollow cylinder, and

wherein the second end of the first elastomeric bumper is attached to the first end surface of the first end of the first rod.

In some preferred embodiments, the elastomer component is contained within the rod, thus preventing possible wear debris from entering the body.

In some preferred embodiments, the elastomer component is contained within the rod within the screw attachment so that the rod can be very short without a central component interfering between the adjacent pedicle screws.

In some preferred embodiments, the elastomer component is hourglass-shaped to provide programmable stiffness in both compression and extension.

### DESCRIPTION OF THE FIGURES

FIG. 1a presents a perspective view of a first embodiment of a polyaxial dynamic stabilization device of the present invention.

FIG. 1b presents a side view of the polyaxial dynamic stabilization device of FIG. 1.

FIG. 1c present a perspective view of an assembly of the polyaxial dynamic stabilization device of FIG. 1a and two bone anchors implanted in a spine model.

FIG. 2a presents a cross-sectional view of the first embodiment of a polyaxial dynamic stabilization device of the present invention in an unloaded condition.

FIG. 2b presents a cross-sectional view of the device of FIG. 2a in extension.

FIG. 2c presents a cross-sectional view of the device of FIG. 2a in flexion.

FIG. 3 presents a cross-sectional view of a second embodiment of a polyaxial dynamic stabilization device of the present invention having a curved rod.

FIG. 4a presents a cross-sectional view of a third embodiment of a polyaxial dynamic stabilization device of the present invention having a pair of elastomeric bumpers.

FIG. 4b presents a cross-sectional view of the device of FIG. 4a, but having a curved rod.

FIG. 5a presents a cross-sectional view of a fifth embodiment of a multi-level polyaxial dynamic stabilization device of the present invention

FIG. 5b presents a cross-sectional view of a sixth embodiment of a multi-level polyaxial dynamic stabilization device of the present invention having a bumpers at its ends.

FIG. 5c presents a cross-sectional view of a seventh embodiment of the multi-level polyaxial dynamic stabilization device of FIG. 5b disposed within a hollow cylinder.

FIG. 6 discloses an embodiment of the present invention having ribber posts.

### DETAILED DESCRIPTION OF THE INVENTION

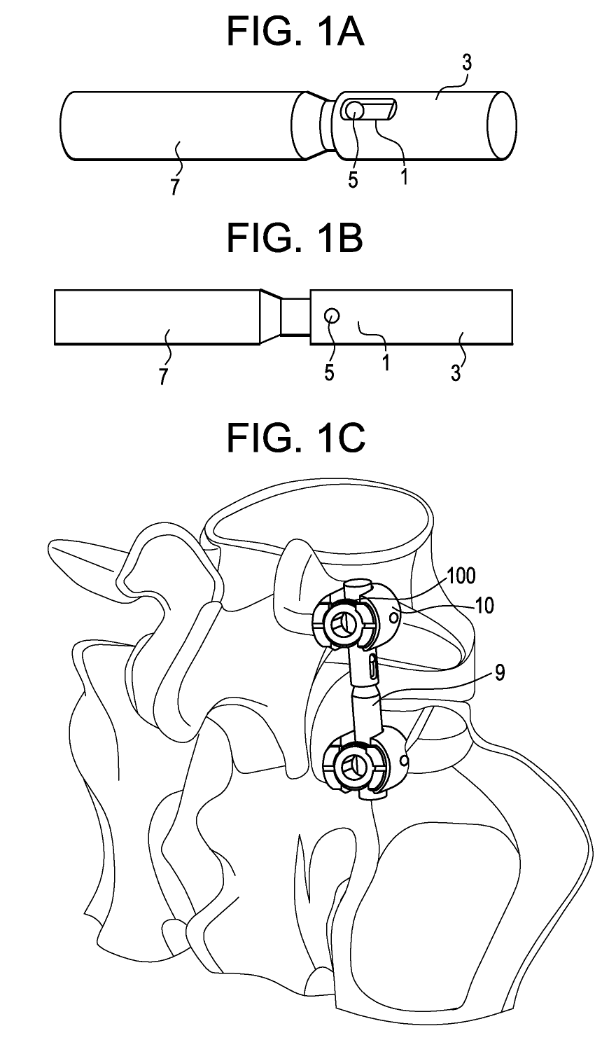

FIGS. 1a-1b show one embodiment of the hollow cylinder-bumper-rod assembly of the present invention including a) a slot 1 in a hollow cylinder component 3 and b) a pin 5 that is fixed to solid rod component 7 and provides controlled motion. FIG. 1c demonstrates how the device 9 may be connected to pedicle screws anchored in the spine by bone anchors 10.

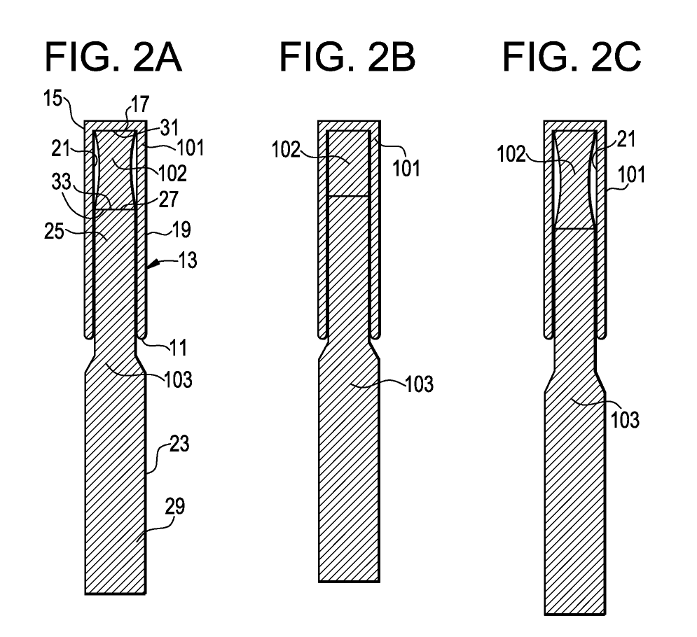

FIGS. 2a-2c demonstrate movement of the device during compression and extension. Now referring to FIG. 2a, there is provided a device in its unloaded state. The device comprises a polyaxial dynamic stabilization device comprising:
a) a first hollow cylinder 101 having an open end 11, an intermediate annular portion 13 and a closed end 15, the closed end defining an inner surface 17, the intermediate annular portion defining an outer annular surface 19 and a first inner annular surface 21,
b) a first rod 103 having an outer diameter 23, a first end 25 having a first end surface 27, and a second end 29, the first end of the first rod being slidably received within the inner annular surface of the first hollow cylinder, and
c) a first elastomeric bumper 102 having a first end 31 and a second end 33,
   wherein the first end of the elastomeric bumper is attached to the inner surface of the closed surface of the first hollow cylinder, and

wherein the second end of the first elastomeric bumper is attached to the first end surface of the first end of the first rod.

The elastomer bumper component 102 preferably has an hourglass shape in an unloaded condition. Both the stiffness of the device and the maximum compression can be controlled by the shape of the hourglass and the type of elastomer chosen. This hourglass shape may be compressed during spine extension until bulging of the middle portion causes the elastomer component to fill the volume within the hollow cylinder above the solid rod end (FIG. 2b). In this compressed condition, the first elastomeric bumper substantially contacts the inner annular surface of the first hollow cylinder (FIG. 2c).

Further, the stiffness of the device in tension (spine flexion) will typically be less than in compression (extended shape shown in FIG. 2c). In this embodiment, the elastomer component is preferably attached at its ends to the hollow cylinder and solid rod component, respectively, so that it may provide resistance to tension. Additionally, the pin component as described in FIG. 1 may provide a limit to the maximum excursion of the device in compression and/or tension.

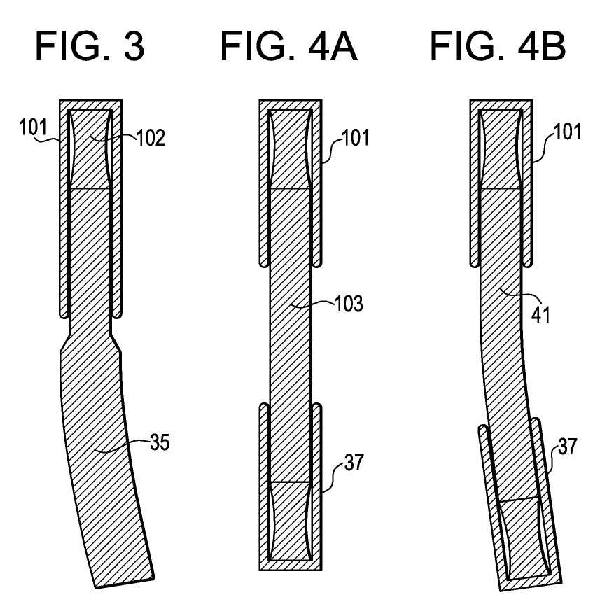

FIG. 3 shows a preferred embodiment where the solid rod component is curved 35 to accommodate lordosis.

FIGS. 4a and 4b show alternate straight and curved embodiments comprising two hollow cylindrical components 101, 37 with two elastomer components 102, 39, respectively, for attachment to adjacent pedicle screws and a straight 103 or curved 41 solid rod component that can slide freely within each of the hollow cylindrical components. This may be preferred when the solid rod component is made of a polymeric material such as PEEK so that the set screw is not clamped onto said material.

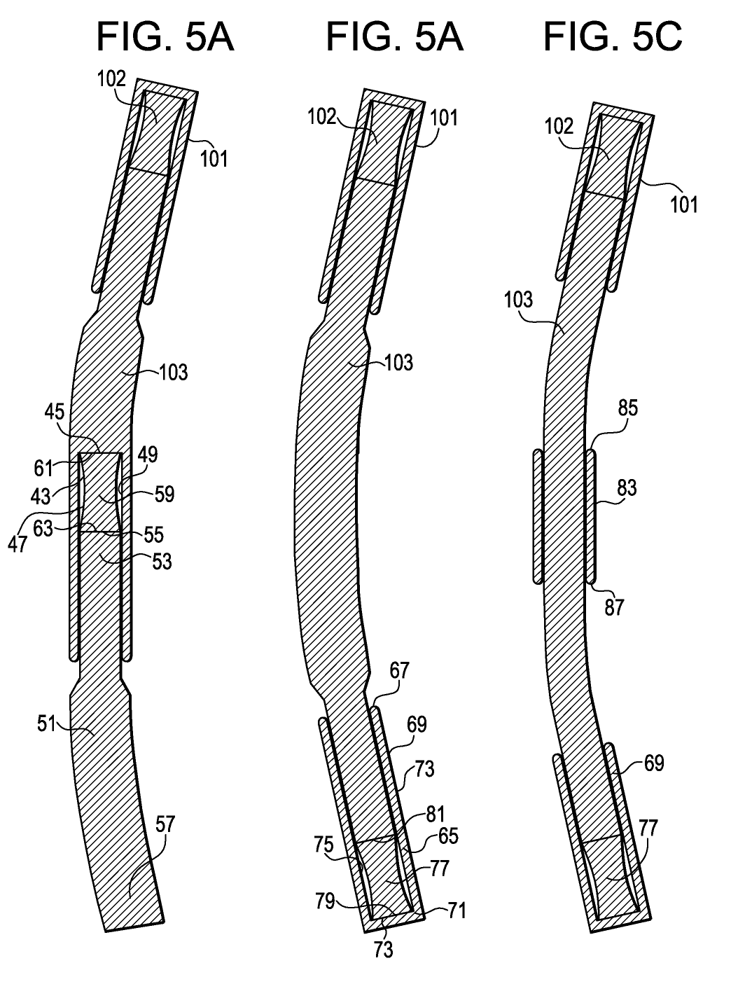

FIGS. 5a-5c shows embodiments of the invention for treating two or more spine levels, i.e. the rod is attached to a first pedicle screw in first vertebral body at the top, a second pedicle screw in second vertebral body in the middle, and a third pedicle screw in third vertebral body at the bottom.

In FIG. 5a, the second end of the first rod forms a blind recess 43 having an blind recess surface 45 and a bore 47 defining a second inner annular surface 49. The device further comprises:
d) a second rod 51 having a first end 53 having a first end surface 55, and a second end 57, the first end of the second rod being slidably received within the second inner annular surface of the first rod, and
e) a second elastomeric bumper 59 having a first end 61 and a second end 63,
   wherein the first end of the second elastomeric bumper is attached to the blind recess surface, and
   wherein the second end of the second elastomeric bumper is attached to the first end surface of the first end of the second rod.
The device of FIG. 5b additionally includes:
d) a second hollow cylinder 65 having an open end 67, an annular portion 69 and a closed end 71, the closed end defining an inner surface 73, the annular portion defining an outer annular surface 73 and an inner annular surface 75,
e) a second elastomeric bumper 77 having a first end 79 and a second end 81,
   wherein the second end of the rod is slidably received within the inner annular surface of the second hollow cylinder,
   wherein the first end of the second elastomeric bumper is attached to the inner surface of the closed surface of the second hollow cylinder, and
   wherein the second end of the second elastomeric bumper is attached to the second end surface of the second end of the rod. In FIG. 5c, the middle of the device comprises a hollow cylinder 83 that is open on both ends 85,87 and is not attached to the solid rod component or an elastomer component but instead provides for unconstrained longitudinal motion between the hollow cylinder and the solid rod. Alternatively, the solid component may be fixed to the central hollow cylinder so that motion is provided only at the end vertebrae. For example, a PEEK solid rod component may be either press fit into or integrally molded to a titanium hollow cylinder. Additionally, this configuration may be appropriate for a single level construct where one end comprises the hollow cylinder, elastomer and solid PEEK rod end and the other end comprises the other solid PEEK rod end press fit or integrally molded into a hollow titanium cylinder. This is particularly beneficial to prevent deformation of the PEEK material from clamping using a typical rod anchor with a set screw. In yet another embodiment, both ends comprise solid PEEK rod ends press fit or integrally molded and the rods are preferably used with a fusion procedure.

In other embodiments of this invention not shown, the device may be integrally connected to a longer rod that is used to fuse the spine at adjacent spine levels at either end. Also, the solid rod component may take a number of alternative forms as described in prior art, such as springs.

One skilled in the art will appreciate that the rod of the device may be configured for use with any type of bone anchor, e.g., bone screw or hook; mono-axial or polyaxial. Typically, a bone anchor assembly includes a bone screw, such as a pedicle screw, having a proximal head and a distal bone-engaging portion, which may be an externally threaded screw shank. The bone screw assembly may also have a receiving member that is configured to receive and couple a spinal fixation element, such as a spinal rod or spinal plate, to the bone anchor assembly.

The receiving member may be coupled to the bone anchor in any well-known conventional manner. For example, the bone anchor assembly may be poly-axial, as in the present exemplary embodiment in which the bone anchor may be adjustable to multiple angles relative to the receiving member, or the bone anchor assembly may be mono-axial, e.g., the bone anchor is fixed relative to the receiving member. An exemplary poly-axial bone screw is described U.S. Pat. No. 5,672,176, the specification of which is incorporated herein by reference in its entirety. In mono-axial embodiments, the bone anchor and the receiving member may be coaxial or may be oriented at angle with respect to one another. In poly-axial embodiments, the bone anchor may biased to a particular angle or range of angles to provide a favored angle the bone anchor. Exemplary favored-angle bone screws are described in U.S. Patent Application Publication No. 2003/0055426 and U.S. Patent Application Publication No. 2002/0058942, the specifications of which are incorporated herein by reference in their entireties.

Therefore, in accordance with the present invention, there is provided a posterior dynamic spinal stabilization system for use in a human spine, comprising:
a) first and second bone anchors, each anchor having a recess for receiving a rod,
b) a first hollow cylinder having an open end, an intermediate annular portion and a closed end, the closed end defining an inner surface, the intermediate annular portion defining an outer annular surface and a first inner annular surface,
c) a first rod having an outer diameter, a first end having a first end surface, and a second end, the first end of the first rod being slidably received within the inner annular surface of the first hollow cylinder, and
d) a first elastomeric bumper having a first end and a second end,
   wherein the first end of the first elastomeric bumper is attached to the inner surface of the closed surface of the first hollow cylinder, and
   wherein the second end of the first elastomeric bumper is attached to the first end surface of the first end of the first rod.
   wherein the outer annular surface of the first hollow cylinder is received in the recess of the first bone anchor,

wherein the second end of the first rod is received in the recess of the second bone anchor.

Generally, in using the present invention, two bone anchors such as polyaxial screws are inserted into adjacent pedicles within a functional spinal unit of a patient. The cylinder-bumper-rod assembly of the present invention is then inserted into the patient between the anchors. The first hollow cylinder is attached to the first bone anchor by laying the outer annular surface of the first hollow cylinder into the first bone anchor recess and tightening an appropriate set screw. Similarly, the second end of the first rod is attached to the second bone anchor by laying the second end into the second bone anchor recess and tightening the appropriate set screw 100 (in FIG. 1c). More preferably, this is achieved in a minimally invasive surgery.

In some embodiments, at least one end of the cylinder-bumper-rod assembly has a bullet nose for ease of insertion.

In some embodiments, the assemble may be implanted in accordance with the minimally invasive techniques and instruments disclosed in U.S. Pat. No. 7,179,261; and US Patent Publication Nos. US2005/0131421; US2005/0131422; US 2005/0215999; US2006/0149291; US2005/0154389; US2007/0233097; and US2005/0192589, the specifications of which are hereby incorporated by reference in their entireties.

Therefore, in accordance with the present invention, there is provided a method of implanting a posterior dynamic spinal stabilization system, comprising the steps of:
a) inserting two bone anchors into adjacent pedicles within a functional spinal unit of a patient, each bone anchor having a recess for receiving a rod,
b) providing a polyaxial dynamic stabilization device comprising:
   i) a first hollow cylinder having an open end, an intermediate annular portion and a closed end, the closed end defining an inner surface, the intermediate annular portion defining an outer annular surface and a first inner annular surface,
   ii) a first rod having an outer diameter, a first end having a first end surface, and a second end, the first end of the first rod being slidably received within the inner annular surface of the first hollow cylinder, and
   iii) a first elastomeric bumper having a first end and a second end,
   wherein the first end of the first elastomeric bumper is attached to the inner surface of the closed surface of the first hollow cylinder, and
   wherein the second end of the first elastomeric bumper is attached to the first end surface of the first end of the first rod,
c) fastening the outer annular surface of the first hollow cylinder into the recess of the first bone anchor, and
d) fastening the second end of the first rod into the recess of the second bone anchor.

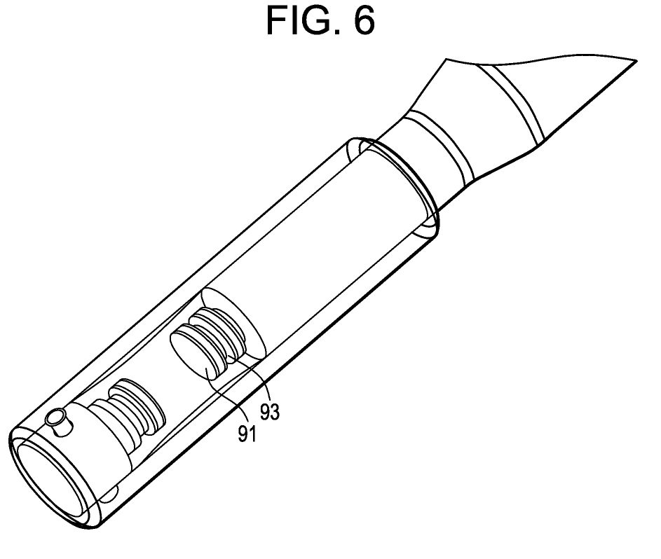

In one preferred embodiment, the geometry of the device components that attach to the elastomer bumper are modified to increase the mechanical interlock and/or to increase the surface area for bonding. Preferably, That is, at least one of the hollow cylinder and the first rod has a predetermined geometry that increases the mechanical interlock with the bumper Now referring to FIG. 6, in one preferred embodiment, ribbed posts 91 are provided both the inner surface of the closed surface of the first hollow cylinder and the first end surface of the first end of the first rod, wherein the post has at least one circumferential groove 93 to increase mechanical interlock with the bumper. Therefore, preferably both the inner surface of the closed surface of the first hollow cylinder and the first end surface of the first end of the first rod have a ribbed post extending therefrom, Preferably, each end of the bumper has a surface that mates with the ribbed post.

In another embodiment, at least one of (and preferably each of) the hollow cylinder and the first rod is mechanically attached to the bumper via tubing that fits over the ends of each of these components. In one such embodiment, the skilled artisan uses this tubing as a “hose” clamp for attaching the inner bumper to the rods or posts, such as PEEK rods. The hose clamp can be used alone or in combination with grooves, posts or bumps on the opposing parts and adhesives depending on the strength requirements. In some embodiments, very thin shrink tubing made from polyester and having a 0.025 mm wall thickness and a 4.35 mm outer diameter is used. In some embodiments, the tubing is obtained from Advanced Polymers Inc, Salem, N.H., as MicroHose Clamps.

Each component of the design may be made from biocompatible, implantable materials known in the art such as stainless steel, titanium, Nitinol, polyetheretherketone (PEEK) or alternative polyarylketones, carbon fiber reinforced polymers, and high performance elastomers such as silicones, dimethylsiloxanes, silicone-urethanes, polyether-urethanes, silicone-polyether-urethanes, polycarbonate urethanes, and silicone-polycarbonate-urethanes.

Preferably, the hollow cylinder components are titanium alloy (Ti-6Al-4V) or cobalt-chrome alloy (e.g. Co—Cr—Mo). If a cobalt-chrome alloy is selected, the alloy is preferably in a work-hardened condition so as to resist deformation upon securing to the bone anchor (e.g with a set screw). Preferably, the solid rod component is either titanium alloy or PEEK. More preferably, the hollow cylinder and solid rod components are selected such that articulation between the two components causes minimal wear, e.g. PEEK solid rod component with titanium alloy hollow cylinder component, or titanium alloy solid rod component with cobalt-chrome hollow cylinder component.

If a metal is chosen as a material of construction, then the metal is preferably selected from the group consisting of nitinol, titanium, titanium alloys (such as Ti-6Al-4V), cobalt-chrome alloys (such as CrCo or Cr—Co—Mo) and stainless steel.

If a polymer is chosen as a material of construction, then the polymer is preferably selected from the group consisting of polycarbonates, polyesters, (particularly aromatic esters such as polyalkylene terephthalates, polyamides; polyalkenes; poly(vinyl fluoride); PTFE; polyarylethyl ketone PAEK; and mixtures thereof.

In some embodiments, the tube and/or solid rod component is made from a composite comprising carbon fiber. Composites comprising carbon fiber are advantageous in that they typically have a strength and stiffness that is superior to neat polymer materials such as a polyarylethyl ketone PAEK. In some embodiments, the tube is made from a polymer composite such as a PEKK-carbon fiber composite.

Preferably, the composite comprising carbon fiber further comprises a polymer. Preferably, the polymer is a polyarylethyl ketone (PAEK). More preferably, the PAEK is selected from the group consisting of polyetherether ketone (PEEK), polyether ketone ketone (PEKK) and polyether ketone (PEK). In preferred embodiments, the PAEK is PEEK.

In some embodiments, the carbon fiber comprises between 1 vol % and 60 vol % (more preferably, between 10 vol % and 50 vol %) of the composite. In some embodiments, the polymer and carbon fibers are homogeneously mixed. In others, the material is a laminate. In some embodiments, the carbon fiber is present in a chopped state. Preferably, the chopped carbon fibers have a median length of between 1 mm and 12 mm, more preferably between 4.5 mm and 7.5 mm. In some embodiments, the carbon fiber is present as continuous strands.

In especially preferred embodiments, the composite comprises:

a) 40-99% (more preferably, 60-80 vol %) polyarylethyl ketone (PAEK), and
b) 1-60% (more preferably, 20-40 vol %) carbon fiber,
wherein the polyarylethyl ketone (PAEK) is selected from the group consisting of polyetherether ketone (PEEK), polyether ketone ketone (PEKK) and polyether ketone (PEK).

In some embodiments, the composite consists essentially of PAEK and carbon fiber. More preferably, the composite comprises 60-80 wt % PAEK and 20-40 wt % carbon fiber. Still more preferably the composite comprises 65-75 wt % PAEK and 25-35 wt % carbon fiber.

The elastomer bumper component is preferably made of a thermoplastic, biocompatible, high performance polycarbonate-urethance (PCU). The stiffness, or durometer of the PCU can be tailored to meet the specifications for the dynamic device. In preferred embodiments, the surface of the device components that will be attached to the elastomer bumper are treated prior to attaching the bumper using known surface treatment methods such as surface roughening (e.g. grit blasting), chemical functionalization (e.g. primers), and plasma treatments know in the art. Alternatively or in conjunction with using a surface treatment, an adhesive may be used to enhance bonding, e.g. using cyanoacrylates. In one preferred embodiment, the surfaces of the device components that will attached to the elastomer bumper will first be roughened using grit blasting, then chemically functionalized using primer, then the elastomer will be overmolded onto the device components.

Also, in some embodiments, the first end of the bumper is unattached. Therefore, also in accordance with the present invention, thee is provided a dynamic stabilization device comprising:
a) a first hollow cylinder having an open end, an intermediate annular portion and a closed end, the closed end defining an inner surface, the intermediate annular portion defining an outer annular surface and a first inner annular surface,
b) a first rod having an outer diameter, a first end having a first end surface, and a second end, the first end of the first rod being slidably received within the inner annular surface of the first hollow cylinder, and
c) a first elastomeric bumper having a first end and a second end,
   wherein the first end of the first elastomeric bumper faces the inner surface of the closed surface of the first hollow cylinder, but is unattached thereto, and wherein the second end of the first elastomeric bumper is attached to the first end surface of the first end of the first rod.

Also, in some embodiments, the second end of the bumper is unattached. Therefore, also in accordance with the present invention, thee is provided a dynamic stabilization device comprising a dynamic stabilization device comprising:
a) a first hollow cylinder having an open end, an intermediate annular portion and a closed end, the closed end defining an inner surface, the intermediate annular portion defining an outer annular surface and a first inner annular surface,
b) a first rod having an outer diameter, a first end having a first end surface, and a second end, the first end of the first rod being slidably received within the inner annular surface of the first hollow cylinder, and
c) a first elastomeric bumper having a first end and a second end,
   wherein the first end of the first elastomeric bumper is attached to the inner surface of the closed surface of the first hollow cylinder, and
   wherein the second end of the first elastomeric bumper faces the first end surface of the first end of the first rod, but is unattached thereto

Also, in some embodiments, the each end of the bumper is unattached. Therefore, also in accordance with the present invention, thee is provided a dynamic stabilization device comprising a dynamic stabilization device comprising:
a) a first hollow cylinder having an open end, an intermediate annular portion and a closed end, the closed end defining an inner surface, the intermediate annular portion defining an outer annular surface and a first inner annular surface,
b) a first rod having an outer diameter, a first end having a first end surface, and a second end, the first end of the first rod being slidably received within the inner annular surface of the first hollow cylinder, and
c) a first elastomeric bumper having a first end and a second end,
   wherein the first end of the first elastomeric bumper faces the inner surface of the closed surface of the first hollow cylinder, but is unattached thereto, and
   wherein the second end of the first elastomeric bumper faces the first end surface of the first end of the first rod, but is unattached thereto.

We claim:

1. A dynamic stabilization device comprising:
a) a first hollow cylinder having an open end, an intermediate annular portion and a closed end, the closed end defining an inner surface, the intermediate annular portion defining an outer annular surface and a first inner annular surface,
b) a first rod having an outer diameter, a first end having a first end surface, and a second end, the first end of the first rod being slidably received within the inner annular surface of the first hollow cylinder, and
c) a first elastomeric bumper having a first end and a second end,
wherein the first end of the first elastomeric bumper is attached to the inner surface of the closed surface of the first hollow cylinder, and
wherein the second end of the first elastomeric bumper is attached to the first end surface of the first end of the first rod.

2. The device of claim 1 wherein the first elastomeric bumper has an hourglass shape in an unloaded condition.

3. The device of claim 2 wherein the first elastomeric bumper substantially contacts the inner annular surface of the first hollow cylinder in extension.

4. The device of claim 1 wherein the rod is curved to provide lordosis.

5. The device of claim 4 wherein the curved rod has a length to span at least two levels of a spine.

6. The device of claim 4 wherein the curved rod has an intermediate portion between the first end and a second end, wherein the intermediate portion is substantially straight.

7. The device of claim 6 wherein the curved rod has a first curve between the first end and the intermediate portion, and a second curve between the second end and the intermediate portion.

8. The device of claim 6 further comprising d) a second hollow cylinder having open ends, wherein the straight intermediate portion of the first rod is slidably disposed within the second hollow cylinder.

9. The device of claim 8, wherein the straight intermediate portion of the first rod is slidably disposed within the second hollow cylinder.

10. The device of claim 8, wherein the straight intermediate portion of the first rod is fixedly disposed within the second hollow cylinder.

11. The polyaxial dynamic stabilization device of claim 1 further comprising:
d) a second hollow cylinder having an open end, an annular portion and a closed end, the closed end defining an inner surface, the annular portion defining an outer annular surface and an inner annular surface,
e) a second elastomeric bumper having a first end and a second end,
wherein the second end of the rod is slidably received within the inner annular surface of the second hollow cylinder,
wherein the first end of the second elastomeric bumper is attached to the inner surface of the closed surface of the second hollow cylinder, and
wherein the second end of the second elastomeric bumper is attached to the second end surface of the second end of the rod.

12. The device of claim 1 further comprising:
d) an extension/compression stop.

13. The device of claim 12 wherein the extension/compression stop comprises i) a slot disposed through the annular portion of the hollow cylinder, and ii) a pin extending from the outer diameter of the rod,
wherein the pin extends through and is slidably received in the slot.

14. The device of claim 1 wherein the second end of the first rod forms a blind recess having an blind recess surface bore defining a second inner annular surface.

15. The device of claim 14 further comprising:
d) a second rod having a first end having a first end surface, and a second end, the first end of the second rod being slidably received within the second inner annular surface of the first rod,

16. The device of claim 15 further comprising:
e) a second elastomeric bumper having a first end and a second end,
wherein the first end of the second elastomeric bumper is attached to the blind recess surface, and
wherein the second end of the second elastomeric bumper is attached to the first end surface of the first end of the second rod.

17. The device of claim 1 having first and second bone anchors attached thereto.

18. The device of claim 1 wherein at least one of the hollow cylinder and the first rod has a predetermined geometry that increases its mechanical interlock with the bumper.

19. The device of claim 18 wherein at least one of the inner surface of the closed surface of the first hollow cylinder and the first end surface of the first end of the first rod has a ribbed post extending therefrom.

20. The device of claim 19 wherein the post has at least one circumferential groove to increase mechanical interlock with the bumper.

21. The device of claim 1 further comprising d) a hose clamp, wherein the hose clamp is attached to i) at least one of the hollow cylinder and the first rod, and ii) at least one end of the bumper.

22. A posterior dynamic spinal stabilization system for use in a human spine, comprising:
a) first and second bone anchors, each anchor having a recess for receiving a rod,
b) a first hollow cylinder having an open end, an intermediate annular portion and a closed end, the closed end defining an inner surface, the intermediate annular portion defining an outer annular surface and a first inner annular surface,
c) a first rod having an outer diameter, a first end having a first end surface, and a second end, the first end of the first rod being slidably received within the inner annular surface of the first hollow cylinder, and
d) a first elastomeric bumper having a first end and a second end,
wherein the first end of the first elastomeric bumper is attached to the inner surface of the closed surface of the first hollow cylinder, and
wherein the second end of the first elastomeric bumper is attached to the first end surface of the first end of the first rod.
wherein the outer annular surface of the first hollow cylinder is received in the recess of the first bone anchor,
wherein the second end of the first rod is received in the recess of the second bone anchor.

23. The system of claim 22 wherein the elastomer bumper is located at least partially within one of the recesses of the bone anchors.

24. A method of implanting a posterior dynamic spinal stabilization system, comprising the steps of:
a) inserting two bone anchors into adjacent pedicles within a functional spinal unit of a patient, each bone anchor having a recess for receiving a rod,
b) providing a polyaxial dynamic stabilization device comprising:
i) a first hollow cylinder having an open end, an intermediate annular portion and a closed end, the closed end defining an inner surface, the intermediate annular portion defining an outer annular surface and a first inner annular surface,
ii) a first rod having an outer diameter, a first end having a first end surface, and a second end, the first end of the first rod being slidably received within the inner annular surface of the first hollow cylinder, and
iii) a first elastomeric bumper having a first end and a second end,
wherein the first end of the first elastomeric bumper is attached to the inner surface of the closed surface of the first hollow cylinder, and
wherein the second end of the first elastomeric bumper is attached to the first end surface of the first end of the first rod,
c) fastening the outer annular surface of the first hollow cylinder into the recess of the first bone anchor, and
d) fastening the second end of the first rod into the recess of the second bone anchor.

25. A dynamic stabilization device comprising:
a) a first hollow cylinder having an open end, an intermediate annular portion and a closed end, the closed end defining an inner surface, the intermediate annular portion defining an outer annular surface and a first inner annular surface,
b) a first rod having an outer diameter, a first end having a first end surface, and a second end, the first end of the first rod being slidably received within the inner annular surface of the first hollow cylinder, and
c) a first elastomeric bumper having a first end and a second end,
wherein the first end of the first elastomeric bumper faces the inner surface of the closed surface of the first hollow cylinder, but is unattached thereto, and
wherein the second end of the first elastomeric bumper is attached to the first end surface of the first end of the first rod.

26. A dynamic stabilization device comprising:
a) a first hollow cylinder having an open end, an intermediate annular portion and a closed end, the closed end defining an inner surface, the intermediate annular portion defining an outer annular surface and a first inner annular surface,
b) a first rod having an outer diameter, a first end having a first end surface, and a second end, the first end of the first rod being slidably received within the inner annular surface of the first hollow cylinder, and
c) a first elastomeric bumper having a first end and a second end,
wherein the first end of the first elastomeric bumper is attached to the inner surface of the closed surface of the first hollow cylinder, and
wherein the second end of the first elastomeric bumper faces the first end surface of the first end of the first rod, but is unattached thereto

27. A dynamic stabilization device comprising:
a) a first hollow cylinder having an open end, an intermediate annular portion and a closed end, the closed end defining an inner surface, the intermediate annular portion defining an outer annular surface and a first inner annular surface,
b) a first rod having an outer diameter, a first end having a first end surface, and a second end, the first end of the first rod being slidably received within the inner annular surface of the first hollow cylinder, and
c) a first elastomeric bumper having a first end and a second end,
wherein the first end of the first elastomeric bumper faces the inner surface of the closed surface of the first hollow cylinder, but is unattached thereto, and
wherein the second end of the first elastomeric bumper faces the first end surface of the first end of the first rod, but is unattached thereto.
